(19) United States
(12) Patent Application Publication
(10) Pub. No.: US 2024/0058502 A1
(43) Pub. Date: Feb. 22, 2024

(54) MEDICAL DEVICE, AND HYDROGEL, PREPARATION METHOD THEREFOR, AND APPLICATION THEREOF

(71) Applicant: GuangZhou Towe Biotech Co., Ltd., Guangzhou (CN)

(72) Inventor: Yue LI, Guangzhou (CN)

(21) Appl. No.: 18/270,396

(22) PCT Filed: Dec. 17, 2021
(86) PCT No.: PCT/CN2021/139083

§ 371 Date: Jun. 29, 2023

(30) Foreign Application Priority Data
Dec. 30, 2020 (CN) 202011613293.2
Mar. 4, 2021 (CN) 202110239606.0
Apr. 15, 2021 (CN) 202110405989.4

(51) Int. Cl. A61L 26/00; A61L 31/04; A61L 31/14; A61L 31/16; C07K 7/06

(52) U.S. Cl. CPC A61L 26/0047; A61L 31/047; A61L 26/0066; A61L 26/008; A61L 31/145; A61L 31/146; A61L 31/16; A61L 26/0085; C07K 7/06; A61L 2300/414; A61L 2300/424

(57) ABSTRACT

Disclosed by the present invention are a medical device, hydrogel, preparation method therefor, and use thereof. The hydrogel is formed by polymerization reaction of antibacterial polypeptide and a buffer solution, the antibacterial polypeptide being polypeptide or a polypeptide derivative thereof represented by the following amino acid sequence: Pro-Phe-Lys-Leu-Ser-Leu-His-Leu-NH₂. The hydrogel of the present invention has self-healing properties, and can be injectable and degraded in vivo and in vitro, needs moderate time for complete degradation, and is degraded after the drug effect is fully achieved; the hydrogel has a remarkable inhibiting effect on growth and proliferation of bacteria and fungi, has antibacterial and anti-inflammatory activity and excellent hemostatic properties, and has the advantages such as small cytotoxicity, substantially expressing no hemolytic activity, and excellent biocompatibility; and the hydrogel according to the present invention is excellent in anti-adhesion activity, does not adhere to wounds, is quickly crosslinked at 37° C., has a excellent effect of preventing postoperative adhesion and has obvious advantages in clinical practice.

### TECHNICAL FIELD OF THE INVENTION

The present invention relates to a hydrogel, in particular to a polypeptide hydrogel, preparation method therefor and use thereof, and medical device suitable for the hydrogel.

### BACKGROUND OF THE INVENTION

Adhesion refers to fibrous bands of scar tissues formed during natural healing process after surgery, physical injury or inflammation, which are usually caused by trauma, bacterial infection and foreign matter residue, etc. during surgery. Regardless of the surgical procedure and location, adhesions develop after almost all operations, of which the incidence of postoperative adhesions in the abdominal and pelvic cavities is about 60%, and that after laparotomy is over 90%. Postoperative adhesions easily lead to intestinal obstruction, female infertility, abdominal pain and other symptoms, and 15-30% patients require a secondary surgery to remove adhesions (namely, an adhesion dissolution). The existence of adhesion will remarkably increase the surgical risk and curative time of patients, which increase the pain and economic burden for patients, so it is urgent to develop an effective postoperative adhesion barrier. However, although there is a great demand for adhesion barriers (particularly in abdominal and cardiothoracic surgeries) in clinic, the use rate of adhesion barriers in practical use is very low, which is less than 10% in abdominal surgeries.

At present, the anti-adhesive materials used in clinic mainly include solid polymer membranes or hydrogels made of polysaccharides and/or (bioresorbable and non-bioresorbable) synthetic polymers, and they play the same role as the physical barrier isolating scar tissues from peripheral organs. The most commonly used commercial anti-adhesive products are mainly used in abdominal surgeries, which are solid bioresorbable membranes composed of hyaluronic acid and carboxymethyl cellulose in the form of films (e.g., Seprafilm, Sanofi/Genzyme) or woven fabrics (e.g., Interceed and Ethicon) (Biomaterials 28 (2007) 975-983). The fact is that it is difficult for these products to completely cover the target tissues and form effective physical barriers. These physical membranes normally fail to effectively prevent the formation of adhesion due to rapid degradation after surgery or detachment with natural movement of tissues. In addition, considering the poor role of the films and fabrics in covering tissues with irregular surfaces or severe folds (for example, great vessels of heart and small intestine, respectively), there is still the risk of adhesion in any uncovered intermediate space.

In order to overcome the shortcomings in the use of solid anti-adhesive membrane, numbers of researches focused on development of sprayable polymer solutions composed of chitosan, hyaluronic acid and/or carboxymethyl cellulose. Although the sprayable polymer solutions are easy to use, they have only a slight effect on preventing the adhesion by reason of short residence time on the injured or inflamed tissues. Moreover, in recent years, although hydrogels formed by in-situ polymerization have been proved to increase their residence time in vivo, the irreversibility of cross-linking in these systems usually makes them fragile or unable to adapt to the dynamic movement of tissues in vivo; and the use of these hydrogels is further constrained by other potential side effects.

Generally, the raw materials for preparing hydrogels are mainly divided into two categories, one is synthetic polymer, and the other is natural biological materials, such as polysaccharide, protein and polypeptide. Wherein, the polypeptide is a compound formed by amino acids jointed together by peptide bonds, which is easily hydrolyzed into amino acids by protease in vivo and has no adverse effects on the body. Therefore, the hydrogel formed with crosslinked polypeptide has excellent biocompatibility and is a promising biomaterial (Adv. Mater. 2017, 1604062).

To sum up, there are still challenges in prevention of postoperative adhesions regardless of numerous materials for the purpose, and it is bound to be a long-term and arduous task to suppress and prevent postoperative adhesion. The development of new anti-adhesive materials based on natural biological polypeptide molecules is an important research direction to prevent the postoperative adhesion and its complications.

### SUMMARY OF THE INVENTION

The objective of the present invention is to provide a medical device, hydrogel, preparation method therefor, and use thereof, and the hydrogel exhibits excellent antibiosis, hemostasis, anti-adhesion and other effects. Based on numerous experimental researches, the inventor of the present invention proves that the hydrogel made from antibacterial polypeptide has such advantages as non-adhesion to the wound surface, self-healing, injectability, temperature sensitivity, antibacterial and hemostatic properties. Besides, the hydrogel has a spatial microstructure capable of loading drugs or growth factors. It may load various drugs or growth factors to realize the functional treatment with dressing and antibacterial and anti-inflammatory functions in wound treatment, and maintain wet environment for the wound surface. In addition, the hydrogel of the present invention is able to be used in combination with various medical devices to achieve more convenient and efficient treatment.

In order to achieve the above-mentioned objectives, the present invention provides a hydrogel, which is formed by polymerization reaction of antibacterial polypeptide and a buffer solution, the antibacterial polypeptide is represented by the following amino acid sequence: Pro-Phe-Lys-Leu-Ser-Leu-His-Leu-NH₂ (953.17 Da).

A derivative or a modification of the above-mentioned amino acid sequence is also suitable for use in the present invention.

The hydrogel of the present invention has a micron-sized porous structure.

A pore size of the micron-sized porous structure of the present invention is 0.05 μm-200 μm.

The buffer solution of the present invention is selected from a group consisting of a carbonate solution, a sulfite solution, a DMEM and a phosphate buffered solution, preferably the phosphate buffered solution; The phosphate buffered solution is prepared by dissolving Na₂HPO₄, KH₂PO₄, KCl and NaCl in deionized water in proportion, wherein the antibacterial polypeptide and the phosphate buffered solution comprises the following components in molar ratio: the antibacterial polypeptide:Na₂HPO₄:KH₂PO₄:KCl:NaCl=(1-40):(1-10):(1-5):(1-5):(50-200), preferably, the antibacterial polypeptide:Na₂HPO₄:KH₂PO₄:KCl:NaCl=(1-40):10:2:2.7:137.

Preferably, the phosphate buffered solution of the present invention further comprises ADP, and a molar ratio of the ADP to Na₂HPO₄ is (1-10):(1-100), more preferably, the molar ratio of the ADP to Na₂HPO₄ is 1:10.

The reaction employed in the present invention may be a physical reaction or a chemical reaction, preferably, an ionic crosslinking polymerization reaction at a reaction temperature of 0° C.-60° C. for a reaction period of 1 min-120 mins.

The present invention also provides a preparation method of the hydrogel, the preparation method of the hydrogel comprises the following steps:
S1, dissolving the antibacterial polypeptide in DMSO to obtain an antibacterial polypeptide solution for further use; and
S2, adding the antibacterial polypeptide solution to a buffer solution, and carrying out an ionic crosslinking polymerization reaction under ultrasonic or stirring conditions to obtain the hydrogel.

A solvent of the hydrogel of the present invention mostly comprises water, which is followed by DMSO, wherein the volume content of the DMSO is less than 5%.

Preferably, the preparation method provided by the present invention further comprises the following step:
S3, adding a drug and/or a growth factor to the buffer solution to obtain the hydrogel loaded with the drug or the growth factor.

Preferably, the drug of the present invention is an antibacterial drug or an anti-inflammatory drug, and the growth factor is a growth factor for promoting wound healing.

The present invention also provides a use of a hydrogel in an anti-adhesion drug, the anti-adhesion drug comprises the hydrogel loaded with a drug or a growth factor and at least one pharmaceutically acceptable carrier and/or excipient.

The anti-adhesion drug of the present invention is in at least one dosage form of tablet, capsule, sugar-coated tablet, granule, drop, spray, rinse, mouthwash, ointment and paste applied on skin surface, and sterile solution for injection. The drug of the present invention is an antibacterial drug or an anti-inflammatory drug, and the growth factor is a growth factor for promoting wound healing.

The hydrogel of the present invention is able to be directly used to wash, spray on, dress or cover the wound surface, or made into a convenient spray that is directly sprayed on the wound surface to form a protective film, thus instantly stopping bleeding, keeping the wound surface moist, creating hypoxic environment conducive to the growth and healing of epithelial cells, and accelerating wound healing. In addition, the antibacterial polypeptide in the hydrogel exhibits a quick-acting and durable broad-spectrum antibacterial effect during wound healing, and is decomposed into metabolizable amino acids after wound healing to avoid adhesion and residue.

In addition, the hydrogel of the present invention can also be used in proper manner and made into the appropriate dosage form according to location of disease or wound. For example, after debridement of wounds, contusions, abrasions, postoperative wounds, burns and scalds, and ulcers, the hydrogel of the present invention is used for spraying and substitution, or wet dressing and bandaging. The hydrogel of the present invention is used for spraying or wet dressing and bandaging on wounds after haemorrhoidectomy, anal abscess excision, anal fistulectomy, fissure excision, neostomy, fistulation, episiotomy and redundant circumcision. The hydrogel of the present invention is used for spraying or wet dressing on topical skin before and after radiotherapy. With respect to chronic non-healing wounds caused by diabetic foot, vasculitis and senile bedsore, the hydrogel of the present invention is used for spraying on the affected sites after debridement. In case of oral odor and postoperative care of oral surgery, the hydrogel of the present invention is made into mouthwash that directly contacts with the oral cavity and is spit out after gargling. The hydrogel of the present invention is used for spraying or wet dressing on surface of a wound of tinea, herpes, acne or the like. The hydrogel of the present invention is used for directly spraying or wet dressing on discomfort, itching, dry or peeling skin caused by skin irritation to improve the skin health.

The hydrogel of the present invention may also be loaded with various drugs or growth factors to realize functional treatment.

Further, the present invention provides a medical device comprising the hydrogel.

The hydrogel of the present invention is coated on at least one surface of the medical device to form a material.

The medical device of the present invention is in the form of any one selected from the group consisting of surgical dressing, fiber, mesh, powder, microsphere, sheet, sponge, foam, suture anchoring device, catheter, stent, surgical tack, plate and screw, drug delivery device, anti-adhesion barrier and tissue adhesive.

The fiber of the present invention is a fabric; the sheet is a membrane or a splint; and the suture anchoring device is a suture or a staple.

The inventor of the present invention found for the first time that the antibacterial polypeptide Pro-Phe-Lys-Leu-Ser-Leu-His-Leu-NH₂ would form the hydrogel. The hydrogel was obtained by an ionic crosslinking polymerization reaction with the antibacterial polypeptide and the phosphate buffered solution as raw materials, and the preparation method for the hydrogel with the antibacterial polypeptide was developed. According to the present invention, the antibacterial polypeptide is applied to the preparation process of the hydrogel, broadening the use scope of the antibacterial polypeptide and producing new types of hydrogel.

In addition, the hydrogel composed of the antibacterial polypeptide of the present invention does not adhere to wounds, and has such advantages as antibacterial activity and hemostatic properties, self-healing, temperature sensitivity, injectability, non-adhesion to cells and no side effect. Further, the hydrogel has the micron-sized porous structure that is able to be used for drug incorporation and controlled-release, such as loading an anti-inflammatory drug, epidermal growth factor or vascular growth factor to accelerate wound healing and reduce the formation of scar tissue fibers.

In addition, the preparation method for the hydrogel of the present invention has the advantages of few steps, convenient process, low requirements for personnel practice and few types of raw materials, thus greatly reducing the production cost. The medical device of the present invention comprises the hydrogel and realizes an efficient treatment in a more convenient way, and the medical device is able to be widely used in clinic.

### DESCRIPTION OF THE DRAWINGS

FIG. 1 shows the images of an antibacterial polypeptide J-1 solution and hydrogels of the present invention;

wherein, A indicates the image of the antibacterial polypeptide J-1 solution; B indicates the image of the hydrogel of Example 1; and C indicates the image of the hydrogel of Example 2.

FIG. 2 shows SEM microstructure images of the hydrogel of Example 2;

wherein, A indicates the SEM image of the antibacterial polypeptide J-1 dissolved in deionized water and dried at room temperature; B indicates the SEM image of the antibacterial polypeptide J-1 hydrogel dried at room temperature; and C indicates the SEM image of the antibacterial polypeptide J-1 hydrogel lyophilized.

FIG. 3 shows a histogram of the inhibitory effects of the hydrogel of the present invention and the control on the proliferations of E. coli, S. aureus and C. albicans.

FIG. 4 shows the growth of E. coli, S. aureus and C. albicans on culture plates with the hydrogels of the present invention.

FIG. 5 shows in vitro degradation property diagrams of the hydrogels of the present invention; wherein, column A shows the histograms of the time required for complete degradation of the hydrogels of the present invention in different pH environment in vitro; and column B shows the mass versus time curves during the in vitro degradation of the hydrogels of the present invention.

FIG. 6 shows the degradation diagrams of the hydrogels of the present invention in mice;

wherein, A indicates the diagram of one mouse after subcutaneous injection of the hydrogel; B-E are B-scan ultrasonic images of mice after 1 day, 3 days, 5 days and 10 days of injection of the hydrogel, respectively.

FIG. 7 shows a histogram of the proliferations of mouse fibroblasts NIH3T3 in different experimental groups.

FIG. 8 shows the hemolytic effect on human red blood cells in different experimental groups.

FIG. 9 shows diagrams of modeling process of postoperative abdominal wall-cecum adhesion in rats of the present invention.

FIG. 10 shows the anti-adhesion effect of the hydrogel of the present invention on the abdominal wall-cecum adhesion model in rats.

FIG. 11 shows histological examination images of the adhesion site one week after treatment with the hydrogel of the present invention.

FIG. 12 shows diagrams of hemostatic effects on liver hemorrhage models in different experimental groups of the present invention;

wherein, 0 s, 60 s and 120 s represent the action duration of the hydrogel.

FIG. 13 shows a histogram of the total bleeding amounts of mouse livers after 120 s in different experimental groups of the present invention.

FIG. 14 shows a histogram of bleeding time of mice in different experimental groups of the present invention.

FIG. 15 shows a flowchart of a preparation method for the hydrogel according to one example of the present invention.

### DETAILED DESCRIPTION OF THE INVENTION

The embodiments of the present invention will be described in detail hereafter in conjunction with the examples. However, those skilled in the art will appreciate that the following examples are only presented for purposes of illustration of the present invention and shall not be construed as restriction to the scope. Antibacterial polypeptide Pro-Phe-Lys-Leu-Ser-Leu-His-Leu-NH₂ used in the examples of the present invention were purchased from Bankpeptide Biological Technology Co., Ltd. and named as antibacterial polypeptide J-1 (purity >95% by HPLC analysis). Unless otherwise specified in the examples, the examples were carried out according to conventional conditions or the conditions recommended by manufacturers. Reagents or instruments used, without specific manufacturers, are conventional products purchased from the market.

1. Preparation of Hydrogel

The hydrogel of the present invention exhibits antibacterial, hemostatic and anti-adhesion effects, and can be used as a medical anti-adhesion hydrogel dressing.

The hydrogel is composed of antibacterial polypeptide Pro-Phe-Lys-Leu-Ser-Leu-His-Leu-NH₂ or its derivative, and the hydrogel has a micron-sized porous structure with a pore size of 0.05 um-200 um.

The hydrogel of the present invention is formed by ionic crosslinking polymerization with antibacterial polypeptide J-1 and a buffer solution, the antibacterial polypeptide J-1 is represented by the following amino acid sequence: Pro-Phe-Lys-Leu-Ser-Leu-His-Leu-NH₂ (953.17 Da). The present invention is not particularly limited to the antibacterial polypeptide J-1, a modification or a derivative of the antibacterial polypeptide J-1 is also suitable for use in the present invention.

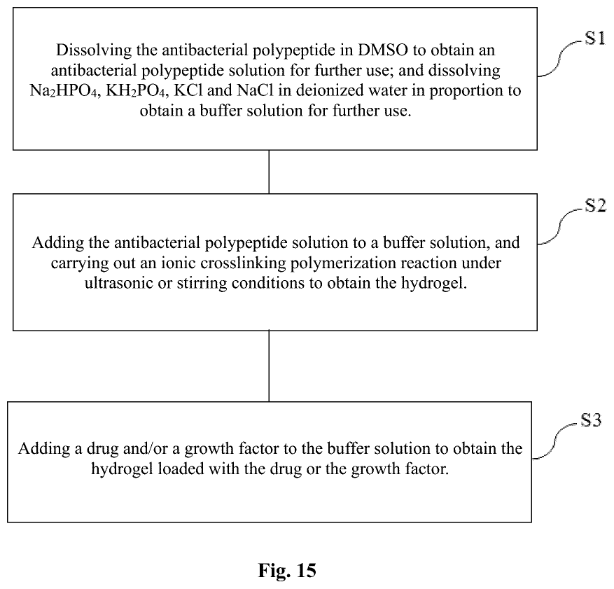

FIG. 15 shows a flowchart of a preparation method for the hydrogel according to one example of the present invention, the preparation method for the hydrogel includes the following steps:
S1, the antibacterial polypeptide J-1 was dissolved in DMSO to obtain an antibacterial polypeptide J-1 solution with a concentration of 100 mM for further use; and Na₂HPO₄, KH₂PO₄, KCl and NaCl were dissolved in deionized water in proportion to obtain phosphate buffered solution for further use.
S2, the antibacterial polypeptide J-1 solution was added to the phosphate buffered solution to a final concentration of 1.5-40 mM, and an ionic crosslinking polymerization reaction was carried out under ultrasonic or stirring conditions to obtain the hydrogel loaded with a drug and/or a growth factor.

Preferably, also including S3, a drug or a growth factor was added to the phosphate buffered solution in the preparation process of the hydrogel in S2 to obtain the hydrogel loaded with the drug or the growth factor.

In S2 of the present invention, the ionic crosslinking polymerization reaction was conducted at a reaction temperature of 0° C.-60° C. for a reaction period of 1 min-120 mins.

In S3 of the present invention, the drug is an antibacterial drug or an anti-inflammatory drug, and the growth factor is a growth factor for promoting wound healing.

Preferably, the phosphate buffered solution of the present invention also includes ADP, and a molar ratio of the ADP to Na₂HPO₄ is (1-10):(1-100). A solvent of the hydrogel of the present invention is mostly composed of water, which is followed by DMSO, the volume content of the DMSO is less than 5%.

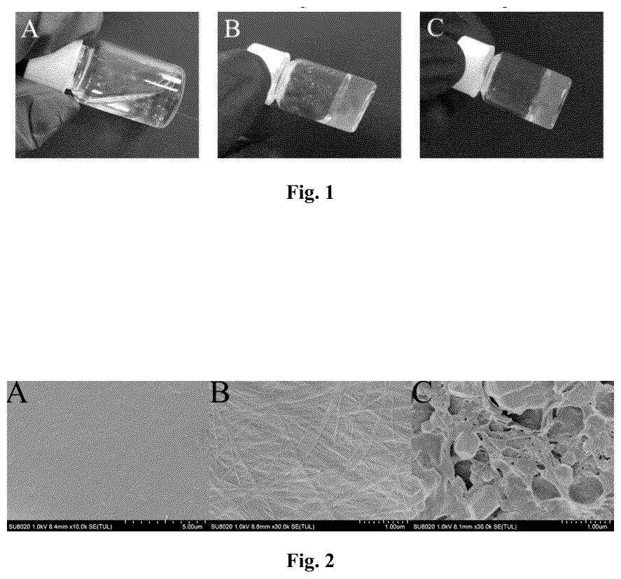

The solution of the antibacterial polypeptide J-1 solution of the present invention is shown as A in FIG. 1. The hydrogel of the present invention can be used in anti-adhesion drugs: an anti-adhesion hydrogel drug is obtained by loading the drug or the growth factor on the hydrogel, and an anti-adhesion hydrogel dressing is obtained by loading the hydrogel on gauze or other implementable carriers.

In order to provide a thorough understanding of the preparation method for the hydrogel of the present invention, the following preferred examples are given for illustration.

### Example 1

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 3:97, and mixed for polymerization at room temperature for 120 mins to obtain the hydrogel.

The hydrogel prepared in the present example was tested to be self-healing and injectable. It was fibrous after drying at room temperature and had micron-sized porous structure after lyophilization. The phase state of the hydrogel is shown as B in FIG. 1.

### Example 2

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 3:47, and mixed for polymerization at room temperature for 120 mins to obtain the hydrogel.

The hydrogel prepared in the present example was tested to be self-healing and injectable. It was fibrous after drying at room temperature and had micron-sized porous structure after lyophilization. The phase state of the hydrogel is shown as C in FIG. 1.

FIG. 2 shows SEM microstructure of the hydrogel of the present example. A indicates the SEM image of the antibacterial polypeptide J-1 dissolved in deionized water and dried at room temperature; B indicates the SEM image of the antibacterial polypeptide J-1 hydrogel dried at room temperature; and C indicates the SEM image of the antibacterial polypeptide J-1 hydrogel lyophilized.

### Example 3

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 1:10, and mixed for polymerization at room temperature for 30 mins to obtain the hydrogel.

The hydrogel prepared in the present example was tested to be self-healing and injectable. It was fibrous after drying at room temperature and had micron-sized porous structure after lyophilization.

### Example 4

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 1:5, and mixed for polymerization at room temperature for 5 mins to obtain the hydrogel.

The hydrogel prepared in the present example was tested to be self-healing and injectable. It was fibrous after drying at room temperature and had micron-sized porous structure after lyophilization.

### Example 5

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 3:97, and mixed for polymerization at 37° C. for 10 mins to obtain the hydrogel.

The hydrogel prepared in the present example was tested to be self-healing and injectable. It was fibrous after drying at room temperature and had micron-sized porous structure after lyophilization.

### Example 6

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 1:47, and mixed for polymerization at 37° C. for 5 mins to obtain the hydrogel.

The hydrogel prepared in the present example was tested to be self-healing and injectable. It was fibrous after drying at room temperature and had micron-sized porous structure after lyophilization.

### Example 7

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 1:10, and mixed for polymerization at 37° C. for 2 mins to obtain the hydrogel.

The hydrogel prepared in the present example was tested to be self-healing and injectable. It was fibrous after drying at room temperature and had micron-sized porous structure after lyophilization.

### Example 8

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (10 mM), KH₂PO₄ (2 mM), KCl (2.7 mM) and NaCl (137 mM) in a volume ratio of 1:5, and mixed for polymerization at 37° C. for 1 min to obtain the hydrogel.

The hydrogel prepared in the present example was tested to be self-healing and injectable. It was fibrous after drying at room temperature and had micron-sized porous structure after lyophilization.

### Example 9

In the present example, a preparation method for a hydrogel includes the following steps: the antibacterial polypeptide J-1 was dissolved in DMSO to obtain a stock solution (100 mM), which was added to a phosphate buffered solution (with pH value adjusted to 6.0-8.0) prepared with Na₂HPO₄ (9 mM), KH₂PO₄ (1.8 mM), KCl (2.43 mM), NaCl (123 mM) and ADP (1 mM) in a volume ratio of 1:5, and mixed for polymerization at 37° C. for 1 min to obtain the hydrogel.

The hydrogel prepared in the present example was tested to be self-healing and injectable. It was fibrous after drying at room temperature and had micron-sized porous structure after lyophilization.

2. Determination of Antibacterial Activity of Hydrogels

The resulting hydrogels obtained by the preparation methods of Example 2 (recorded as the hydrogel 1) and Example 9 (recorded as the hydrogel 2) were used as test samples, and the strains employed in the antibacterial activity test were gram-negative bacterium E. coli (ATCC 25922), gram-positive bacterium S. aureus (ATCC 29213) and fungus C. albicans (ATCC 14053). Mueller-Hinton (MH) medium was used for two bacteria and Sabouraud dextrose (SD) medium was used for the fungus. During the test, 200 uL of the antibacterial polypeptide hydrogel was added to a 1.5 mL Eppendorf tube, 400 uL of the bacterial solution (1*10⁶ cfu/mL) was carefully added on the hydrogel in the tube, and then cultured in a shaker (at 120 rpm) at 37° C. After 24 hours of incubation, the supernatant was obtained to measure OD₆₀₀. A histogram was plotted with the OD₆₀₀ value of the bacterial solutions from tubes as ordinate, and PBS was used as the control.

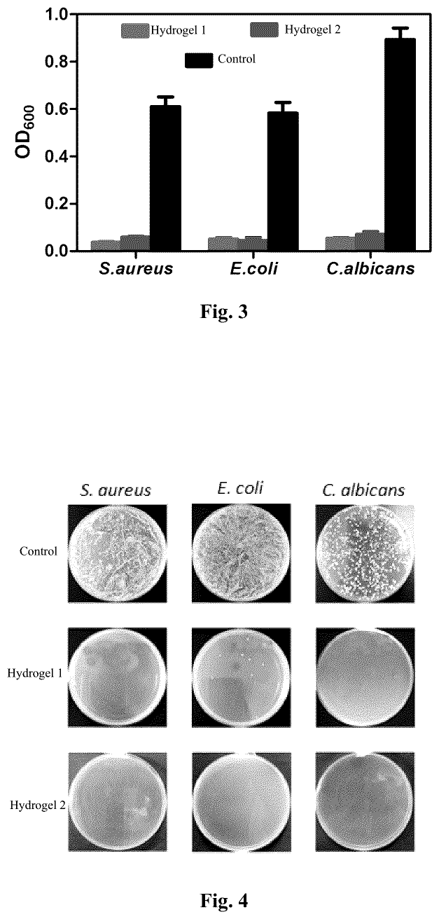

FIG. 3 shows a histogram of the inhibitory effects of the hydrogels of the present invention and the control on the proliferations of E. coli, S. aureus and C. albicans. As shown in FIG. 3, compared with the control group, the hydrogel 1 and the hydrogel 2 significantly inhibited the proliferations of the test bacteria and fungus; Before measuring the OD value, 100 uL of bacterial suspensions were taken from tubes, diluted properly, spread uniformly on a prepared culture plate, respectively, and then cultured at 37° C. overnight. Refer to FIG. 4, which shows the growth of E. coli, S. aureus and C. albicans on culture plates with the hydrogels of the present invention. As shown in FIG. 4, the culture plates in the control group are full of bacterial cells, while no growth of E. coli, S. aureus or C. albicans is observed on the culture plates with the hydrogel 1 and the hydrogel 2.

It can be seen that the hydrogels of the present invention exhibit a significant inhibiting effect on the growth and proliferations of the bacteria and the fungus.

3. Determination of In Vitro and In Vivo Degradations of Hydrogels Determination of In Vitro Degradation:

The resulting hydrogels obtained by the preparation methods of Example 2 (recorded as the hydrogel 1) and Example 9 (recorded as the hydrogel 2) were used as test samples, the in vitro degradation of the hydrogels was carried out as follows: 200 uL of the hydrogel was added to a 1.5 mL EP tube weighed in advance, 200 μL of PBSs with pH values of 6.4, 7.4 and 8.4 was added on the hydrogel, respectively, cultured in an incubator at 37° C. for 24 hours, and then the mass of the residual hydrogel was recorded after removing the solution on the hydrogel with a pipet; and another 200 μL of PBS was added to the EP tube and incubated again until the hydrogel was completely degraded. Then histograms were plotted with the time for complete decomposition of the hydrogel in different pH environment tubes as ordinate.

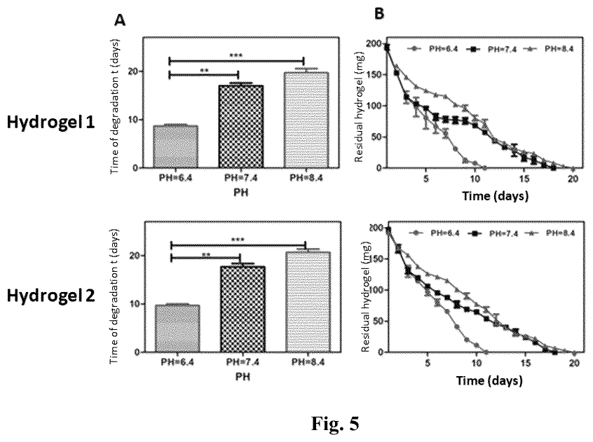

FIG. 5 shows in vitro degradation property diagrams of the hydrogels of the present invention; wherein, column A shows the histograms of the time required for complete degradation of the hydrogel 1 and the hydrogel 2 in different pH environment in vitro; and column B shows the mass versus time curves during the in vitro degradation of the hydrogel 1 and the hydrogel 2. Refer to column A in FIG. 5, the hydrogel 1 and the hydrogel 2 were completely degraded after 10 days in the presence of the PBS buffer solution with pH value of 6.4, and after 18 days and 20 days in the presence of PBS buffer solutions with pH values of 7.4 and 8.4, separately. Refer to column B in FIG. 5, graphs were plotted with the degradation days as abscissa and the hydrogel mass as ordinate, it can be seen that the degradations of the hydrogel 1 and the hydrogel 2 show linear correlation with time.

Determination of In Vivo Degradation:

The resulting hydrogels obtained by the preparation methods of Example 2 (recorded as the hydrogel 1) and Example 9 (recorded as the hydrogel 2) were used as test samples to determine the in vivo degradation of the hydrogels. Compared with the in vitro environment, the in vivo environment is more complicated and subject to the influence of various tissue fluids, enzymes and animal exercises. The degradation of hydrogel in animals was determined by subcutaneous injection of the hydrogel in mice, which quickly restored to gel at the injection site, and the residual hydrogel in subcutaneous tissue of the mice was detected by B-scan ultrasonography.

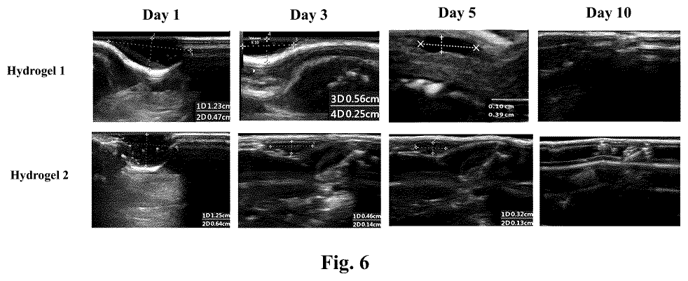

FIG. 6 shows the degradation diagrams of the hydrogels of the present invention in mice; from left to right are the B-scan ultrasonic images of the hydrogel 1 and the hydrogel 2 after 1, 3, 5 and 10 days of injection, respectively. It can be seen these images, the hydrogel 1 and the hydrogel 2 are progressively degraded in animals and substantially degraded after 10 days.

It can be seen that the hydrogels of the present invention have self-healing properties, and are injectable and degraded in vivo and in vitro, need moderate time for complete degradation, and are degraded after the drug effect is fully achieved.

4. Determination of Biocompatibility of Hydrogels

The resulting hydrogels obtained by the preparation methods of Example 2 (recorded as the hydrogel 1) and Example 9 (recorded as the hydrogel 2) were used as test samples. The biocompatibility of the hydrogels of the present invention was evaluated by analyzing their toxicity to mammalian cells (mouse fibroblasts NIH3T3 were used in the test) and hemolytic activity on human red blood cells.
(1) Specifically, the toxicity to mammalian cells was determined by MTT colorimetry including the following steps: 100 uL of a hydrogel was added to a 96-well plate in advance, 100 uL of DMEM was carefully added on the hydrogel, equilibrated for 24 hours, the DMEM was removed with a pipet, and then 5000 cells (100 uL) were seeded to each well and incubated in a cell incubator for 24 hours, MTT was added and incubated for another 4 hours, the supernatant was discarded, 150 uL of DMSO was added to each well to dissolve the formazan fully, and then measured using an ELISA reader (OD₅₇₀). Regarding to the positive control group, normal saline solutions containing the same concentration of the antibacterial polypeptide J-1 as the hydrogel 1 and the hydrogel 2 were used as a solution 1 and a solution 2, DMEM was used in the negative control group, and these groups shared the other steps of the experimental method.

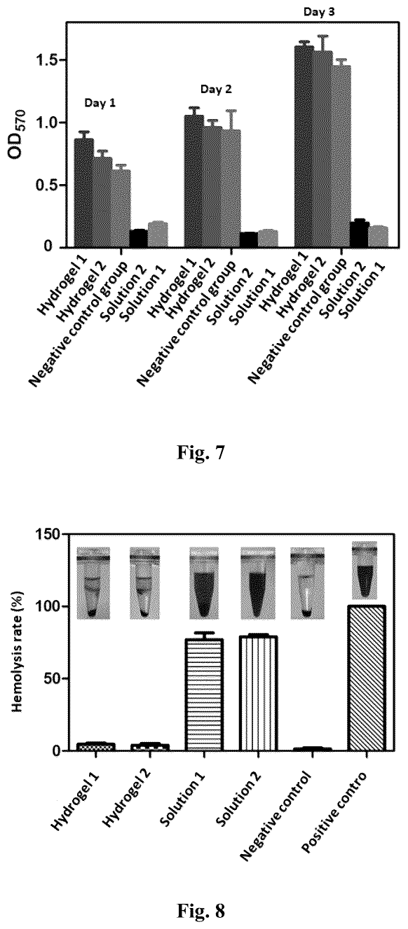

FIG. 7 shows a histogram of the proliferations of mouse fibroblasts NIH3T3 in different experimental groups. As shown in FIG. 7, the proliferations of mouse fibroblasts NIH3T3 are substantially the same between the hydrogel 1 and the hydrogel 2 treatment group wells and the negative control wells, showing extremely low cytotoxicity.
(2) To measure the hemolytic activity of the hydrogel on human red blood cells, the hydrogel 1, the hydrogel 2, the solution 1 (normal saline solution containing the same concentration of the antibacterial polypeptide J-1 as the hydrogel 1), the solution 2 (normal saline solution containing the same concentration of the antibacterial polypeptide J-1 as the hydrogel 2), PBS (negative control group) and 2% Triton (positive control group) (200 uL each) were added to corresponding 1.5 mL EP tubes, and 800 uL of 8% human red blood cells was added to these EP tubes respectively and incubated for 1 hour in an incubator at 37° C., centrifuged at 1200 g, and then photographed to observe the release of haemoglobin. The supernatant was taken from each tube to measure OD₄₉₀, and then the hemolysis rate was calculated quantitatively.

FIG. 8 shows the hemolytic effect on human red blood cells in different experimental groups. It can be seen from the results FIG. 8, the hydrogel of the present invention substantially exhibits no hemolytic activity.

It can be seen that the hydrogels of the present invention have small cytotoxicity, substantially express no hemolytic activity, and have excellent biocompatibility.

5. Anti-Adhesion Activity of Hydrogel on Abdominal Wall-Cecum Injury-Induced Adhesion Model in Rats

The resulting hydrogels obtained by the preparation methods of Example 2 (recorded as the hydrogel 1) and Example 9 (recorded as the hydrogel 2) were used as test samples. In the anti-adhesion effect test with a hydrogel dressing after surgery, only clean SD rats were used, which were single-cage raised at the temperature of 22° C.-24° C. and the relative humidity of 45%-55%, and fasted 12 hours before operation.

Modeling of abdominal wall-cecum injury-induced adhesion: Each rat was anesthetized by intraperitoneal injection of 3 mg/mL pentobarbital sodium at a dose of 1 mL/100 g of body weight, positioned on a heated operating table, with hypoabdominal skin shaved, disinfected and covered with towel, and then a 5-cm incision was made along the center line of the hypoabdominal skin. The right abdominal wall was held with a pair of hemostatic forceps, an area with a depth of about 0.5 mm and a size of about 1 cmx 2 cm was cut with a scalpel at a distance of about 1 cm from the central incision in the abdominal wall, and then the superficial muscle in this area was detached with a pair of ophthalmic scissors to form a bleeding wound. After that, the surface of the cecum corresponding to the wound surface of the abdominal wall was gently rubbed with a surgical brush until the serosa layer of the cecum was destroyed and had visible punctate bleeding. As a result, the abdominal wall-cecum defects were produced. The mesentery of the cecum was sutured at the upper right corner of the abdominal wall wound with a 30 gauge suture to ensure full contact between the wound surfaces of the abdominal wall and the cecum. The rats were divided into a control group and hydrogel treatment groups (with 6 rats in each group) and interfered correspondingly. Finally, the muscle layer and the skin layer of the abdominal wall were sutured continuously for abdominal wound closure with a 4-0 gauge suture. All operations were carried out under aseptic conditions.

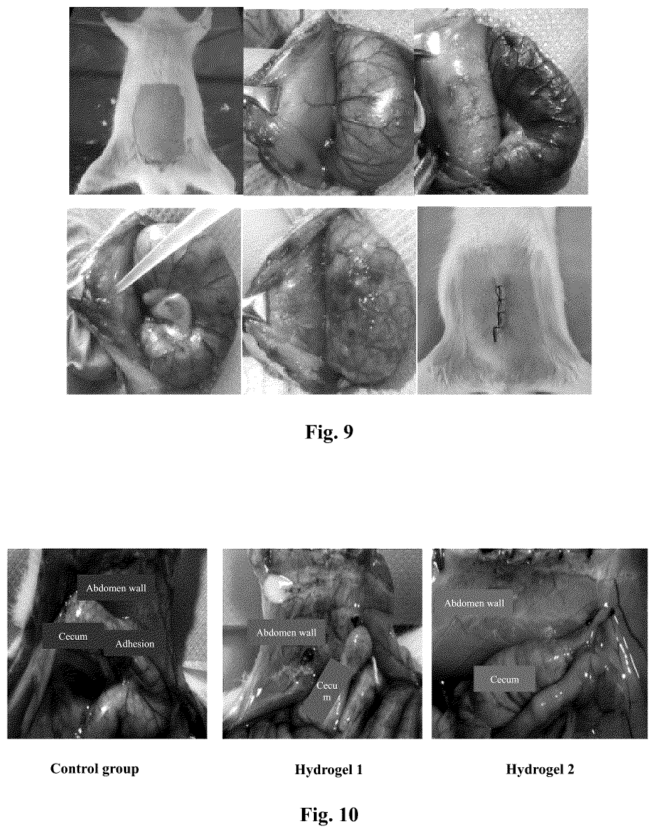

During the operation, the rats in the control group were washed with normal saline, and the rats in the hydrogel treatment groups were given 2 mL of the hydrogel that was uniformly applied on the wounds, separately. Seven days after the operation, all rats of the control group underwent laparotomy and exhibited dense abdominal wall-cecum adhesion (see FIG. 9), and almost all of them obtained an adhesion score of 5 points. No adhesion was observed in rats of the hydrogel 1 treatment group and the hydrogel 2 treatment group, and the abdominal wall wound healed well. The slight light-colored scar was remarkably smaller than the initial wound, and the cecum basically returned to normal, with some slight scratches (see FIG. 10), obtaining an adhesion score of 0 point. An adhesion occurred between the surgical incision and the cecum rather than wound surfaces in one hydrogel group.

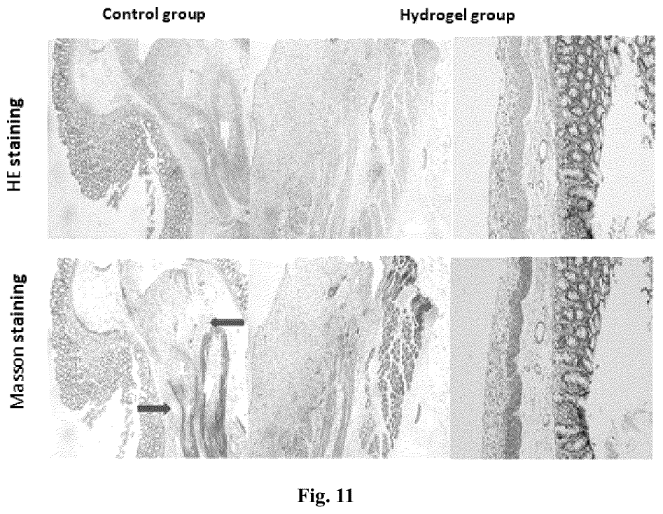

The adhesion tissues were analyzed 7 days after the operation. For rats of the control group, the HE staining results showed that the abdominal wall and cecum were connected by dense tissue adhesion, and the Masson staining results revealed a large number of collagen fibers in the adhesion area (refer to FIG. 11). In the hydrogel 1 treatment group and the hydrogel 2 treatment group, the wounds in the abdominal wall and the cecum of rats recovered well without adhesion. Clear and evenly distributed neo-mesothelial layers were observed on the wound surfaces, with some inflammatory cells infiltrated into the wounds. The Masson staining showed fibrosis tissues to different extents beneath the mesothelial cell layer (see FIG. 11).

It can be seen that the hydrogel of the present invention has excellent anti-adhesion activity.

6. Determination of Hemostatic Properties of a Hydrogel in a Mouse Liver Hemorrhage Model

The resulting hydrogels obtained by the preparation methods of Example 2 (PBS+peptide hydrogel group) and Example 9 (ADP+peptide hydrogel group) were used as test samples. In the hemostatic property test with a hydrogel, only male Kunming mice (18 g-22 g of body weight) were used, which were raised at the temperature of 22° C.-24° C. and the relative humidity of 45%-55%, and fasted 12 hours before operation.

Establishment of liver hemorrhage modeling: In the experiment, mice were divided into three groups: a control group, a PBS+peptide hydrogel group and an ADP+peptide hydrogel group, with 8 mice in each group. Each mouse was anesthetized at a dose of 40 mg/kg of body weight, positioned on an operating table with abdominal skin shaved, and the operative site was disinfected with iodophor. A longitudinal incision with a diameter of about 1.5 cm was made in the abdomen, and tissues were detached layer by layer to fully expose the right lobe of liver. A piece of filter paper weighed in advance was placed under the right lobe of liver, which was then punctured in the center with a 21 G needle. After that, 200 uL of the hydrogel was immediately applied on the wound (the control group was free from any treatment), and the site was photographed to record the liver hemorrhage process. The bleeding time of liver was recorded. After the experiment, the filter paper was taken out and weighed to calculate the amount of bleeding.

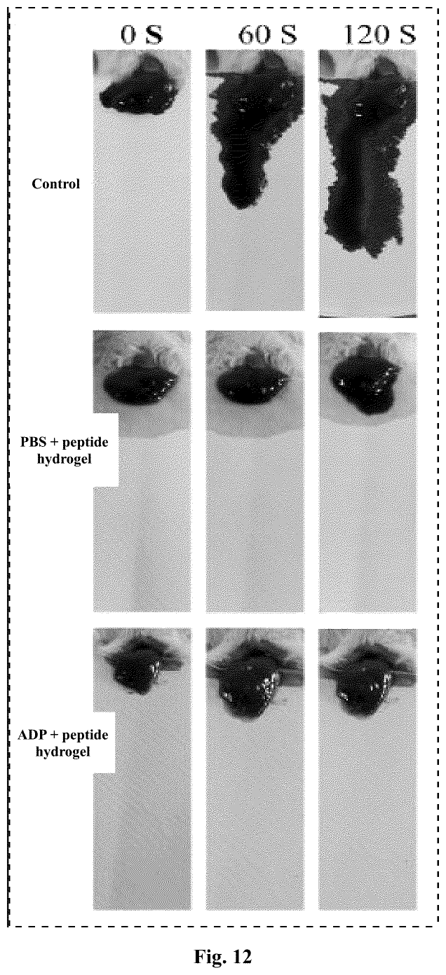

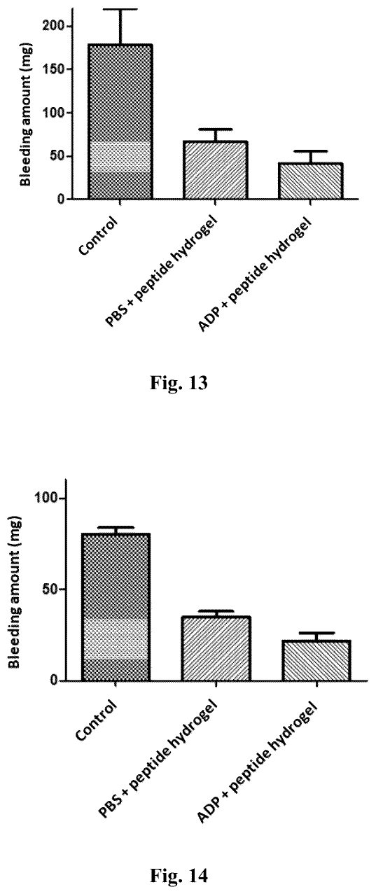

FIG. 12 shows hemostatic effects on the liver hemorrhage models in different experimental groups of the present example; wherein, 0 s, 60 s and 120 s represent the action duration of the hydrogel. As shown in FIG. 12, the PBS+peptide hydrogel group and the ADP+peptide hydrogel group have significant hemostatic effects compared with the control group without any treatment. FIG. 13 shows a histogram of the total bleeding amount (after 120 s) of mouse liver in different experimental groups of the present example. According to FIG. 13, the bleeding amounts of mouse liver are ranked as: the ADP+peptide hydrogel group, the PBS+peptide hydrogel group, the control group in an ascending order. Refer to FIG. 14, which shows a histogram of the bleeding time of mice in different experimental groups of the present example. According to FIG. 14, the mouse liver completely stopped bleeding 25 s after using the ADP+peptide hydrogel, 40 s after using the PBS+peptide hydrogel, and 85 s without any treatment in the control group.

It can be seen that the hydrogel of the present invention has excellent hemostatic effect.

In conclusion, the hydrogel of the present invention has self-healing properties, and is injectable and degraded in vivo and in vitro, needs moderate time for complete degradation, and is degraded after the drug effect is fully achieved; the hydrogel has a remarkable inhibiting effect on the growth and proliferation of bacteria and fungi, has antibacterial and anti-inflammatory activity and excellent hemostatic properties, and has the advantages such as small cytotoxicity, substantially expressing no hemolytic activity, and excellent biocompatibility; and the hydrogel is excellent in anti-adhesion activity, does not adhere to wounds, is quickly crosslinked at 37° C., has a excellent effect of preventing postoperative adhesion and has obvious advantages in clinical practice.

It will be understood that the present invention is not intended to be limited to the above description of the preferred examples. All modifications and deformations made by those skilled in the art without departing from the spirit of the present invention can be incorporated in the protection scope of the appended claims of the present invention.

1. A hydrogel, wherein the hydrogel is formed by polymerization reaction of antibacterial polypeptide and a buffer solution, the antibacterial polypeptide being polypeptide or a polypeptide derivative thereof represented by the following amino acid sequence: Pro-Phe-Lys-Leu-Ser-Leu-His-Leu-NH₂.

2. The hydrogel according to claim 1, wherein the hydrogel has a micron-sized porous structure.

3. The hydrogel according to claim 2, wherein a pore size of the micron-sized porous structure is 0.05 μm-200 μm.

4. The hydrogel according to claim 1, wherein the buffer solution is a phosphate buffered solution; and the antibacterial polypeptide and the phosphate buffered solution comprises the following components in molar ratio: the antibacterial polypeptide:Na₂HPO₄:KH₂PO₄:KCl:NaCl=(1-40):(1-10):(1-5):(1-5):(50-200).

5. The hydrogel according to claim 4, wherein the phosphate buffered solution further comprises adenosine diphosphate (ADP), and a molar ratio of the ADP to Na₂HPO₄ is (1-10):(1-100).

6. The hydrogel according to claim 4, wherein the reaction is an ionic crosslinking polymerization reaction at a reaction temperature of 0° C.-60° C. for a reaction period of 1 min-120 mins.

7. The hydrogel according to claim 1, wherein a preparation method of the hydrogel comprises the following steps:
S1, dissolving the antibacterial polypeptide in dimethyl sulfoxide (DMSO) to obtain an antibacterial polypeptide solution for further use; and
S2, adding the antibacterial polypeptide solution to a buffer solution, and carrying out an ionic crosslinking polymerization reaction under ultrasonic or stirring conditions to obtain the hydrogel.

8. The hydrogel according to claim 7, wherein further comprising the following step:
S3, adding a drug and/or a growth factor to the buffer solution to obtain the hydrogel loaded with the drug or the growth factor.

9. The hydrogel according to claim 8, wherein the drug is an antibacterial drug or an anti-inflammatory drug, and the growth factor is a growth factor for promoting wound healing.

10. The hydrogel according to claim 7, wherein the volume content of the DMSO is less than 5%.

11. The hydrogel according to claim 1 in an anti-adhesion drug, wherein the anti-adhesion drug comprises the hydrogel loaded with a drug and/or a growth factor and at least one pharmaceutically acceptable carrier and/or excipient.

12. The hydrogel according to claim 11, wherein the anti-adhesion drug is in at least one dosage form of tablet, capsule, sugar-coated tablet, granule, drop, spray, rinse, mouthwash, ointment and paste applied on skin surface, and sterile solution for injection.

13. The hydrogel according to claim 11, wherein the drug is an antibacterial drug or an anti-inflammatory drug, and the growth factor is a growth factor for promoting wound healing.

14. A medical device, wherein the medical device comprises the hydrogel according to claim 1.

15. The medical device according to claim 14, wherein the hydrogel is coated on at least one surface of the medical device to form a material.

16. The medical device according to claim 14, wherein the medical device is in the form of any one selected from the group consisting of surgical dressing, fiber, mesh, powder, microsphere, sheet, sponge, foam, suture anchoring device, catheter, stent, surgical tack, plate and screw, drug delivery device, anti-adhesion barrier and tissue adhesive.

17. The medical device according to claim 16, wherein the fiber is a fabric; the sheet is a membrane or a splint; and the suture anchoring device is a suture or a staple.
